(19) United States
(12) Patent Application Publication
(10) Pub. No.: US 2003/0068386 A1
(43) Pub. Date: Apr. 10, 2003

(54) Method of treating cancerous disease

(76) Inventor: Detcho A. Stoyanovsky, New York, NY (US)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 09/947,310

(22) Filed: Sep. 5, 2001

(51) Int. Cl. A61K 33/00; A61K 31/66; A61K 31/46; A61K 31/21; A61K 31/16

(52) U.S. Cl. 424/600; 514/509; 514/575; 514/304; 514/142

(57) ABSTRACT

This invention discloses a method of treating a patient having a cancerous disease comprising administering to the patient an effective amount of a composition capable of generating a hydroxyl radical, wherein the extracellular pH of the cancer cells is less than 7.0. Preferably, the method includes employing N-substituted hydroxyl amines as the composition capable of generating the hydroxyl radical.

### BACKGROUND OF THE INVENTION

1. Field of the Invention

This invention relates to a method of treating a patient having a cancerous disease (carcinoma) including administering to the patient a compound capable of generating a hydroxyl radical.

2. Brief Description of the Background Art

Treatment of malignant tumors and metastases of malignant tumors to other locations of a patient's body presents a challenge in effecting destruction of the malignant tumors while leaving normal healthy cells unharmed. Chemotherapy is a systemic treatment generally based upon cell destruction of both cancerous cells and normal healthy cells. Chemotherapy, prior to the present invention, is a non-specific treatment affecting all proliferating cells, whether normal or malignant. Heretofore, chemotherapy approaches for the treatment of patents having a cancerous disease often involved undesirable side effects to other vital organs of the patient's body such as for example, bone marrow cell destruction, and kidney damage. Further, patients undergoing chemotherapy experience unwanted side effects such as for example, but not limited to, diarrhea, nausea and loss of body hair.

The present invention provides a method of treating a patient with cancer comprising administering to the patient a compound capable of generating a hydroxyl radical. For example, administration to a patient of sodium trioxodintrate (Na₂N₂0₃) otherwise known by those skilled in the art as Angeli's salt (AS) under acidic conditions is toxic to malignant human fibroblasts, but is not toxic to malignant human fibroblasts under non-acidic conditions.

It is known by those skilled in the art that the intense metabolism of glucose to lactic acid and the hydrolysis of adenosine triphosphate (ATP) in hypoxic tumor regions lead to acidification of the microenvironment in tumor tissues. In actively glycolyzing tumors, the extracellular pH is approximately 6.2, versus a pH of about 7.4 of the extra and intracellular milieu of normal cells. Further reduction in the extracellular pH may be achieved in some tumors by administration of glucose (+/−insulin) and by drugs, such as for example, hydralazine, which modify the relative blood flow to tumors and normal tissues. The tumor pH gradient is used in the present invention for targeting of cancer tissue, as most anticancer drugs must be transported either via active transport or by passive diffusion into the cells. Since all of these processes may be pH-dependent, the cytotoxic activity of an anticancer drug may depend on its pKa, and on both the extra-and intracellular pH of the targeted cell.

In past model studies aimed to mimic the biochemistry of the nitroxyl anion (NO⁻) sodium trioxodintrate is often used as a specific NO⁻ donor. (See H. H. Schmidt, etal., Proc. Natl. Acad. Sci. U.S.A., Vol. 93, pages 14492-7 (1996); H. Ohshima, etal., Free Radic. Biol. Med., Vol. 26, pages 1305-13 (1999); and M. N. Hughes, et al., Methods Enzymol. Vol. 301, pages 279-87 (1999)).

Analytical Chemistry, Vol. 71, No. 3, pages 715-721 (1999), Detcho A. Stoyanovsky, et al., describes the electron spin resonance (ESR) and high performance liquid chromatography with electrochemical (HPLC-EC) analysis of the interaction of the hydroxyl radical with dimethyl sulfoxide (DMSO) and the spin-trapping of the methyl radical with nitrones to form stable nitroxides. Stoyanovsky, et al., show that in an alpha (4-pyridyl-1-oxide)-N-tert-butylnitrone (POBN) and alpha-phenyl-N-tert-butylnitrone (PNB) spin-trapping aimed at detecting methyl radical in biological systems, the nitroxides formed can be reduced to their respective ESR hydroxylamine derivatives.

Molecular Medicine Today, Vol. 6, pages 15-19 (2000), M. Stubbs, et al., set forth that tumor cells have a lower extracellular pH than normal cells.

Proc. Natl. Acad. Sci. USA, Vol. 96, pages 14617-14622 (1999) X. L. Ma, et al., describes the effects of nitric oxide and the nitroxyl anion in myocardial ischemia and reperfusion injury. Ma, et al., sets forth that the nitroxyl anion increases tissue damage that occurs during ischemia and reperfusion.

The Journal of Pharmacology and Experimental Therapeutics, Vol. 293, No. 2, pages 545-500 (2000) K. M. K. Boje, et al., describe the effects of nitric oxide redox species (NO., NO⁺, NO⁻) on the permeability alterations of the blood-brain barrier.

### BRIEF DESCRIPTION OF THE DRAWINGS

FIG. 1, “A” shows electron spin resonance spectra of NO.—Fe^{II}-MGD formation at pH 6.0, and “B” shows the effects of the proton concentration on the AS-dependent formation of NO.—Fe^{II}-MGD.

FIG. 2 shows electron spin resonance spectra (1-6) of DMPO, POBN, and PBN nitroxides formed in a solution of AS.

FIG. 3, “A” shows electron spin resonance spectra of PBN plus AS in the presence of DMSO, “B” shows HPLC-EC profile of a solution of AS, PBN and DMSO, and “C” HPLC-UV (ultraviolet) profile of a solution of AS, DMSO, and POBN.

FIG. 4 shows the optimization of the POBN-dependent spin trapping.

FIG. 5 shows the effects of the proton concentration on the HO. generation that parallels the hydrolysis of AS, and the AS-induced toxicity to normal human fibroblasts and human breast cancer cells.

FIG. 6 shows the electron spin resonance spectra of PBN/CH₃.

### SUMMARY OF THE INVENTION

The present invention has met the hereinbefore described needs. The present invention provides a method for treating a patient having a cancerous disease comprising administering to the patient an effective amount of a composition capable of generating a hydroxyl radical (HO.) in a pH dependent manner. The present invention further includes providing a method for treating a patient having a cancerous disease comprising administering to the patient an effective amount of a composition capable of generating a hydroxyl radical in a pH dependent manner via the intermediate formation of a nitroxyl anion. More specifically, the method of the present invention includes administering the composition to the patient wherein the extracellular pH of the cancer cells is less than 7.0, and preferably between about 4-6.5.

The method of the present invention includes administering to the patient a composition that is an N-substituted hydroxylamine having a formula: X—NY—OH, wherein X is an electron withdrawing group and Y is selected from the group consisting of —H, CH₃CO—, and —CO—O′—CO—NH. Preferably, the electron-withdrawing group is selected from the group consisting of —NO₂, (EtO)₂P(O)—, —SO₂—, and C₆H₅SO₂.

The method of the present invention includes administering to the patient a composition that has the formula R₂C═N(O)—OH (nitronates), and wherein R is preferably selected from one or more of the groups consisting of an alkyl and an aryl residues.

In another embodiment of the present invention, the method includes incorporating the composition, as described herein, in a suitable pharmaceutical carrier and administering a therapeutically effective amount of a composition incorporated into the pharmaceutical carrier to the patient.

Further, the present invention provides a method including employing the composition, as described herein, in prophylactically treating a patient to provide protection against a cancerous illness.

In another embodiment of the present invention, a method is provided for inhibiting the growth of a cancerous tumor in a patient comprising administering to the patient a composition capable of generating a hydroxyl radical in an amount effective to inhibit the growth of the cancerous tumor.

### DETAILED DESCRIPTION OF THE INVENTION

The present invention provides a method for treating a patient having a cancerous disease comprising administering to the patient an effective amount of a composition capable of generating a hydroxyl radical in a pH-dependent manner.

As used herein, “effective amount” means that amount necessary to bring about a desired effect, such as for example, inhibition of malignant cancerous cells.

As used herein, “metastasis” is the transfer of malignant tumor cells, or neoplasm, via the circulatory or lymphatic systems or via natural body cavities of a patient, usually from the primary focus of neoplasia to a distant site in the body of the patient, and subsequent development of secondary tumors or colonies in the new location.

As used herein, “metastases” means the secondary tumors or colonies formed as a result of a metastasis.

As used herein, “inhibition of metastasis” is defined as preventing or reducing the development of metastases.

As used herein, “patient” means one or more members of the animal kingdom including but not limited to, human beings.

The present invention provides a method for treating a patient having a cancerous disease comprising administering to the patient an effective amount of a composition that is capable of generating a hydroxyl radical and wherein the extracellular pH of the cancer cells is less than 7.0. The method of the present invention preferably includes wherein the composition that is capable of generating a hydroxyl radical is an organic compound with activated-N—O— function(s) such as, for example but not limited to, an N-substituted hydroxylamine, nitronate, ester of hydroxamic acid, P-nitrosophosphate, 2-Oxa-3-aza-bicyclo[2.2.2]octane derivative, which hydrolyze with release of NO⁻. The composition employed in the method of the present invention have the following formula: X—N—(Y)—OH and X₂—N—OH, wherein X is an electron withdrawing group and Y is selected from the groups consisting of —H, —O—, CH₃CO—, and CO—O′—CO—NH. Preferably, X is —NO₂, (EtO)₂P(O)—, SO₂—, and C₆H₅SO₂.

In another embodiment of the method of the present invention, the composition has the formula R₂C═N(O)—OH, wherein R is selected from one or more of the groups consisting of an alkyl, or aryl residue. In another embodiment of the method of the present invention, the composition is hyponitrous acid (HO—N═N—OH).

Although not wishing to be bound by any particular theory, the present inventor proposes that the composition of the present invention inhibits malignant cell growth and metastasis according to one or more of the following mechanisms. NO⁻ (or its protonated form, HNO) can trigger a production of hydroxyl radical (HO.) via the intermediate formation of HO—N═N—OH followed by an azo-type decomposition, well known by those skilled in the art, in a pH-dependent manner.

For example, a pronounced (i.e. 50 times higher) Angeli's salt (AS, sodium trioxodinitrate, donor of NO⁻ )-dependent production of HO. is observed within the pH interval of about 4-6.5, the latter coinciding with the extracellular pH of malignant tumor cells, as compared to the minor production of HO. at pH 7.4.

Due to its high energy, HO. is one of the most toxic species that could be formed in a biological system. When generated, HO. will react with a neighboring molecule at first collision, i.e., it cannot diffuse from its site of generation to a distance greater than that to the nearest molecule (diffusion-controlled reaction). As HO. is generated in the vicinity of the plasmatic membrane of a tumor cell (pH˜6.2), it will destroy the tumor cell. HO. has minimal chance of diffusing to a healthy cell as the inter-cellular space is filled with numerous low molecular weight compounds and proteins that are able to intercept this species. At physiological pH (7.4; normal cells), the AS (NO⁻) dependent production of HO. is negligible (Scheme I).

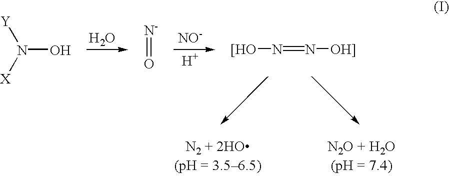

Generally, X (as described herein) is any electron-withdrawing group that makes up an N-derivatized hydroxylamine that may promote a pH-dependent formation of a hydroxyl radical, for example a polar N-substituted hydroxylamine, and Y is as described herein. The present invention also includes a method for treating a patient, having a cancerous disease comprising administering to the patient an effective amount of a composition capable of generating a hydroxyl radical (HO.) wherein the composition is nitric oxide plus a nitric oxide-reducing compound. Nitric oxide may be reduced to its one electron reduction product nitroxyl anion (NO⁻). It is postulated herein that NO⁻ is a unique pH sensor targeting cancerous cells in the method of the present invention.

The method of the present invention includes incorporating the composition, as described herein, in a suitable pharmaceutical carrier and administering a therapeutically effective amount of the composition incorporated into the pharmaceutical carrier to the patient. The method of the present invention includes administering the composition, as described herein, incorporated into the pharmaceutical carrier, as described herein to the patient by such as for example, but not limited to, the oral, parenteral, subcutaneous, transdermal, and topical routes. Further, for example, the composition of the instant invention incorporated into the pharmaceutical carrier may be administered to the patient by intracavity administration such as by injection into the location of the cancerous disease. The method for treating a patient having a cancerous disease of the present invention includes administering the composition, as described herein, via the intracavitary route. For example, administering the composition of the instant invention directly into a body cavity such as for example, the peritoneal cavity, the plural cavity and the cavities of the central nervous system.

The pharmaceutical carrier of the present invention may be any pharmaceutical carrier known by those skilled in the art such as for example, sterile water for injection, physiologic saline, 5% dextrose for injection, 5% NaHCO₃, and combinations thereof.

The method of the present invention includes employing the method in prophylactically treating a patient to provide protection against a cancerous illness.

In a further embodiment of the present invention, a method is provided for inhibiting the growth of a cancerous tumor in a patient comprising administering to the patient a composition capable of generating a hydroxyl radical in an amount effective to inhibit the growth of a cancerous tumor. As discussed herein, cancer is an invasive disease and tends to metastasize to new sites. It spreads by spreading directly into surrounding tissues and also may be disseminated through the lympathic and circulatory systems. The exact cause of cancer in human beings is unknown. Unregulated, disorganized proliferation of cell growth may be caused for example by various forms of chronic irritation, certain agents and by viruses. Cancer may affect almost any organ or part of the body.

### MATERIALS AND METHODS

The hydrolysis of N-hydroxylamine derivatives is paralleled by the generation of hydroxyl radicals. For example, sodium trioxodinitrate (AS) undergoes a hydrolysis reaction that most likely follows a reaction mechanism that includes the recombination of NO⁻ to cis-hyponitrous acid (Na₂N₂0₂), which undergoes an azo-type homolytic fission with release of HO. and as described in Journal of the American Chemical Society, Vol. 121, No. 21, pages 5093-5094 (1999), Detcho A. Stoyanovsky et al., reaction Scheme II below.

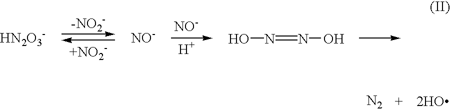

Depending on the degree of protonation, the stability of AS in aqueous solutions follows the sequence N₂0₃²⁻>HN₂0₃>H₂N₂0₃ (pK₁=3.0 and pK₂=9.35 as set forth in J. Chem. Soc., Dalton, pages 703-706 (1976), M. N. Hughes, et al. AS is relatively stable in alkaline solutions. Its rate of decomposition within the pH interval of 3.5 to 8.5 is rapid, proton-independent and proceeds via an intermediate formation of NO⁻ and NO₂⁻ and N₂0 as end products, Id. (see reaction Scheme III below). At lower pH values, the decomposition rate increases with increasing acidity with production of NO..

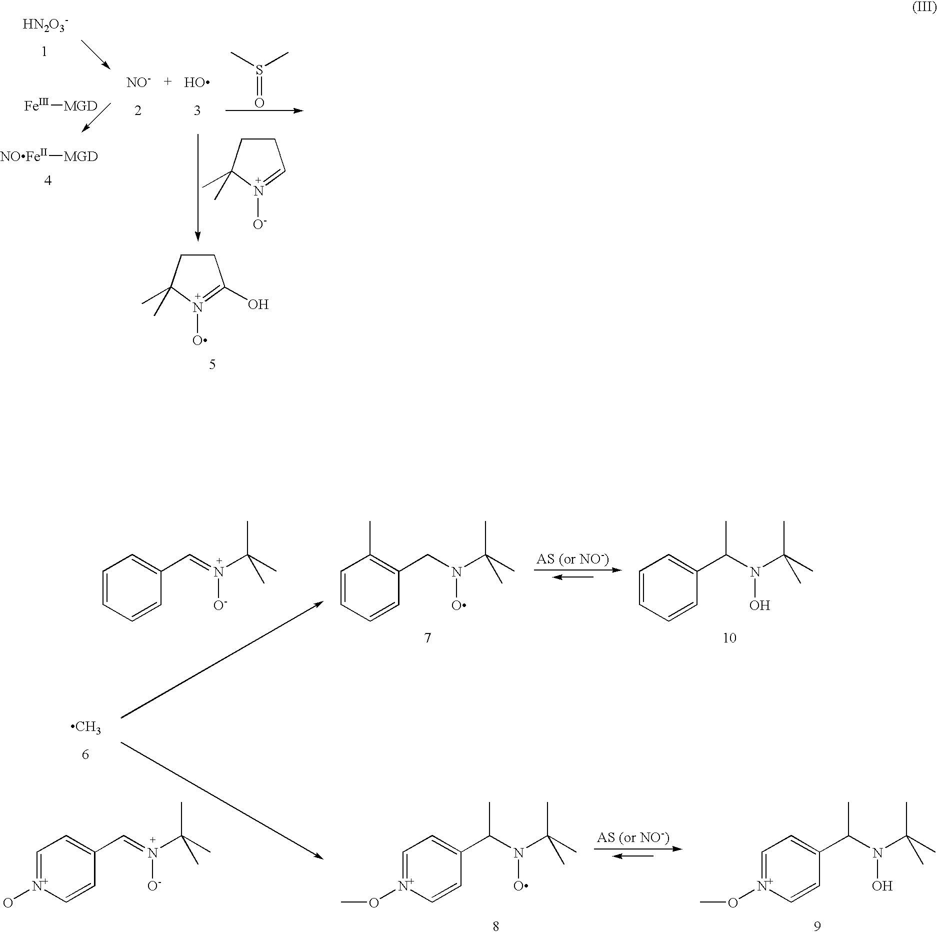

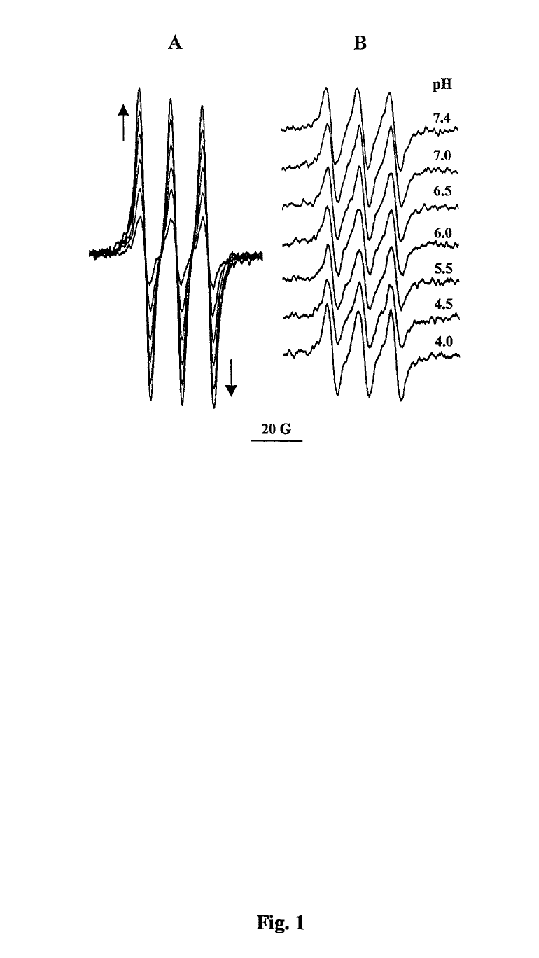

Free Radic. Biol. Med., Vol. 29, pages 793-797 (2000) Y. Yia, et al., and Free Radic. Biol. Med., Vol. 27, pages 347-355 (1999), K. Tsuchiya, et al., set forth that when AS hydrolysis is carried out in the presence of Fe³⁺ and N-methyl-D-glucamine dithiocarbarnate (MGD), the characteristic ESR spectrum of NO.—Fe^{II}-MGD formed via the interaction of NO. and Fe^{III}-MGD was observed (see reaction Scheme III, (2) to (4), and FIG. 1A).

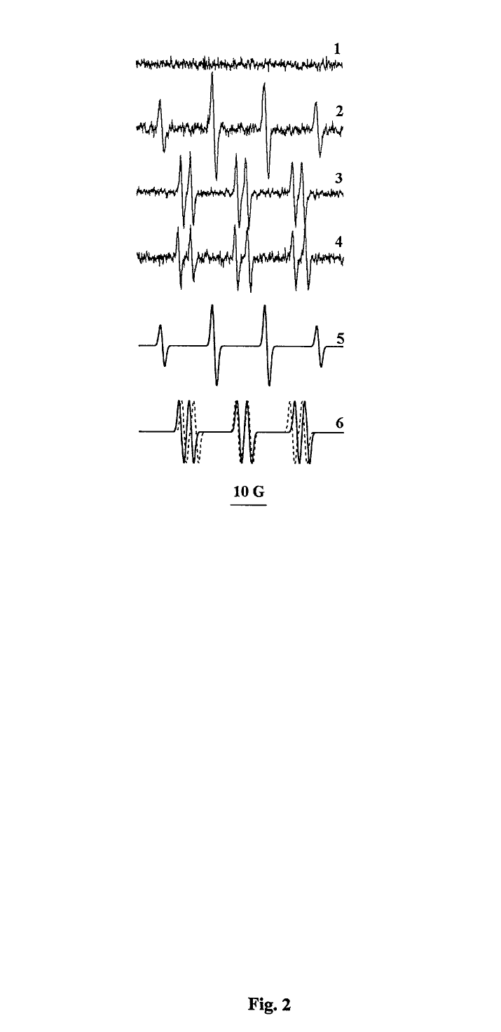

In FIG. 1, the ESR spectra of NO.—Fe^{II}-MGD formed in solutions of AS is shown. All ESR spectra were recorded in 0.1 M Tris buffer (at 20° C.) containing AS (0.1 M), MGD (1 mM) and FeCl₃ (0.3 Mm). FIG. 1, “A” shows the ESR-monitored kinetics of NO.—Fe^{II}-MGD formation at pH 6.0. The time interval between two consecutive ESR scannings was 5 minutes. FIG. 1, “B” shows the effects of the proton concentration on the AS-dependent formation of NO.—Fe^{II}-MGD. Within the pH interval of 3.5 to 7.4, the formation of NO.—Fe^{II}-MGD was relatively constant (FIG. 1B), suggesting that the rate of NO⁻ (nitroxyl anion) generation was proton-independent. A substitution of NO—Fe^{II}-MGD with 5,5′-dimethyl-1-pyroline N-oxide (DMPO) resulted in the appearance of a four-line ESR spectra with hyperfine structure (in Gauss) of a_{N}=15.0 and a_{H}=15.0 which allows the assignment of the adduct as that formed by addition of HO. to DMPO as set forth in reaction Scheme III, (3 to 5), and FIG. 2. ESR spectra of DMPO, POBN and PBN nitroxides formed in a solution of AS. ESR measurements and spectra simulations were performed as described in Analytical Chemistry, Vol. 71, No. 3, pages 715-721 (1999), Stoyanovsky, et al. DMPO, POBN and PBN were used at concentrations of 0.12, 0.10 and 0.05 M, respectively. All reactions were carried out in phosphate buffer (pH 7.4; 20° C.). The hyperfine splitting constants (in Gauss) used for simulation of the spectra of the DMPO/.OH, POBN/.CH₃ and PBN/.CH₃ nitroxides were as follows: (a_{N}=14.9; a_{H}=14.9), (a_{N}=16.10; a_{H}=2.77), and (a_{N}=16.46; a_{H}=3.36), respectively. AS was synthesized as described in Am. Chem. Soc., Vol. 82, pages 5731-5740 (1960), P. A. S. Smith, et al. ESR spectra were recorded after 5 minutes of incubation of the reaction solutions in the absence or in the presence of 0.5 mM AS (a stock solution of AS was prepared in 0.2 M NaOH). FIG. 2, spectrum 1 shows DMPO; spectrum 2 shows DMPO plus AS; spectrum 3 shows POBN plus AS plus DMSO (0.2 mM), spectrum 4 shows PBN plus AS plus DMSO (0.2 mM); trace 5 represents simulation of the ESR spectrum of DMPO/.OH; trace 6 represents computer simulation of the ESR spectra of POBN/.CH3 (solid lines) and PBN/.CH3 (dashed lines). The ESR spectra in repetitive experiments did not differ more than 10% (n≧3). Chelators of metal ions (e.g. EDTA and Chelex 100), catalase (500-2500U/ml) and superoxide dismutase (30-3000 U/ml) did not affect the AS-dependent formation of reaction Scheme III, 5, suggesting that transition metal ions, H₂0₂ and superoxide anion are not involved in this production (data not shown). The maximal, steady state spin concentration of reaction Scheme III, 5 was 5.4 μM (1% from the initial concentration of AS) as determined by double integration of the ESR signal using 4-hydroxyl-1-TEMPO as a standard as set forth in “Spin Labeling Theory and Applications”, B. L. J., Ed (Academic Press, Orlando, Fla.) 1976. Since AS can generate ONOO— under aerobic conditions that may affect the ESR spin-trapping measurements, experiments under anaerobic conditions were carried out. Removal of oxygen from the reaction solutions, however, did not decrease the ESR spectra of reaction Scheme III, 5, suggesting that the formation of the nitroxide reflects a HO.— rather than ONOO⁻-dependent oxidation of DMPO. The AS-dependent generation of HO. was further supported by determination of the rate constant for the interaction of HO. with DMPO as described in J. Biol. Chem., Vol. 263, pages 1204-1211 (1988), K. M. Morehouse, et al. The present investigator obtained a value of 3.52×10⁹ M⁻¹s⁻¹, which is in good agreement with the known literature values of 2.1-3.4×10⁹ 10⁹ M⁻¹s⁻¹.

The low stability of .OH-derived nitroxides is a limiting factor for quantitation of .OH. The latter experimental difficulty could be partly solved with the introduction of dimethylsulfoxide (DMSO) into the studied systems. .OH oxidizes DMSO to methyl radical .CH₃, shown in reaction Scheme III, 3 to 6, which forms relatively stable nitroxides with alpha-(4-Pyridyl-1-oxide)-N-tert-butylnitrone (POBN), shown in reaction Scheme III, 6 to 8, and alpha-phenyl-N-tert-butylnitrone (PBN) shown in reaction Scheme III, 6 to 7 that can be quantified by HPLC with electrochemical and/or UV detection, see Analytical Chemistry, Stoyanosky, et al., supra. Thus, the hydrolysis of AS in the presence of DMSO and either POBN or PBN produced the typical ESR spectra of reaction Scheme III, 7 and 8, respectively (FIG. 2, trace 3-POBN/.CH₃; trace 4-PBN/.CH₃). The “10 G” stands for the dimension of the ESR spectra; G=Gauss; and describes the magnetic field and used for measuring the hyperfine structure of the ESR spectra The computer simulated ESR spectra of reaction Scheme III, 5, 7 and 8 were in good agreement with the experimental spectra (FIG. 2, traces 5 and 6, respectively).

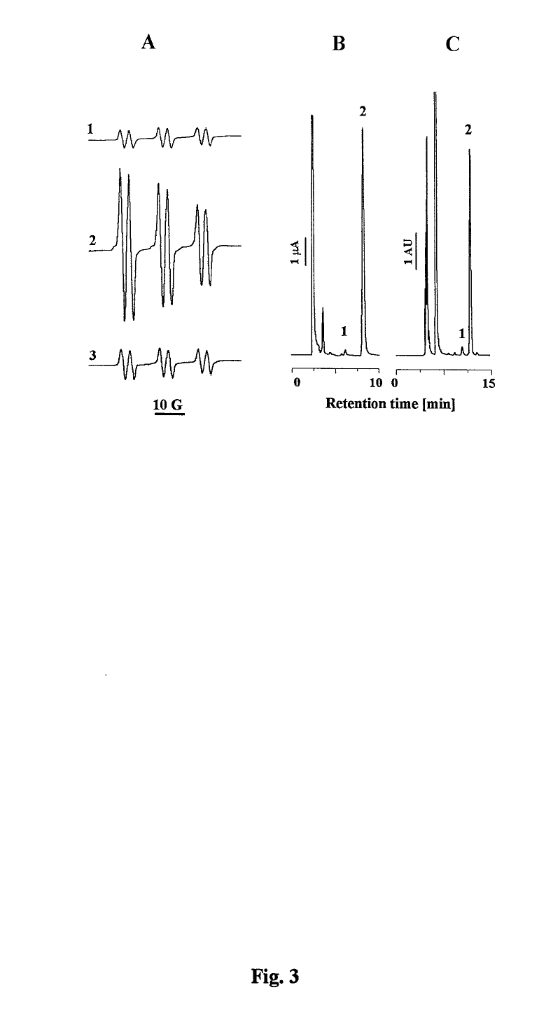

It is known that both AS and NO⁻ can act as reductants, which suggests that the nitroxides of reaction Scheme III, 5, 7 and 8 may not reflect the real amount of the spin-trapped .HO (reaction Scheme III, 3) and .CH₃ (reaction Scheme III, 6). In the presence of reductants, most nitroxides are in equilibrium with the corresponding ESR silent hydroxylamines. The latter assumption is supported by the ESR spectra presented in FIG. 3A.

FIG. 3 shows the ESR and HPLC-EC/UV analyses of PBN/.CH₃ and POBN/.CH₃ nitroxides and hydroxylamines formed in solutions of DMSO and AS. All experiments were carried out in 0.1 M phosphate buffer (pH=6.0) containing DMSO (0.2 M) and either PBN (0.05 M) or POBN (0.1 M). FIG. 3, A, Spectrum 1 shows PBN plus AS (1 mM); after an incubation of 20 minutes at 20° C., K₃[Fe(CN)₆] (0.5 mM) was added and consecutive ESR spectra were recorded after 2 minutes shown as spectrum 2, and 4 minutes shown in spectrum 3, respectively. FIG. 3, B, shows the HPLC-EC profile of a solution of AS, DMSO and PBN after an incubation of 30 minutes at 37° C. (centigrade). The chromatographic separation was achieved on a C18 matrix (Column-Microsorb, 4.6 mm×25 cm, 5μ, Rainin Instrument Company, Inc., Emeryville, Calif.) with a mobile phase consisting of 70% methanol and 20 mM LiC10₄ at a flow rate of 1 ml per min. (milliliter per minute). Electrochemical detection was carried out at +0.8 volts. Peaks 1 and 2 of FIG. 3B show the elution of PBN/CH₃ nitroxide and hydroxylamine, respectively. FIG. 3, C, shows the HPLC-UV profile of a solution of AS, DMSO and POBN after an incubation of 30 minutes at 37° C. All chromatographic conditions were as indicated in FIG. 3B, except that instead of PBN, POBN was used, and the detection of analytes was carried out at 261 nanometers (nm) with a Shimadzu photodiode array detector (SPD-M10AVP, Princeton, N.J.). FIG. 3, C, peaks 1 and 2 reflect the elution of POBN/CH₃ nitroxide and hdroxylamine, respectively.

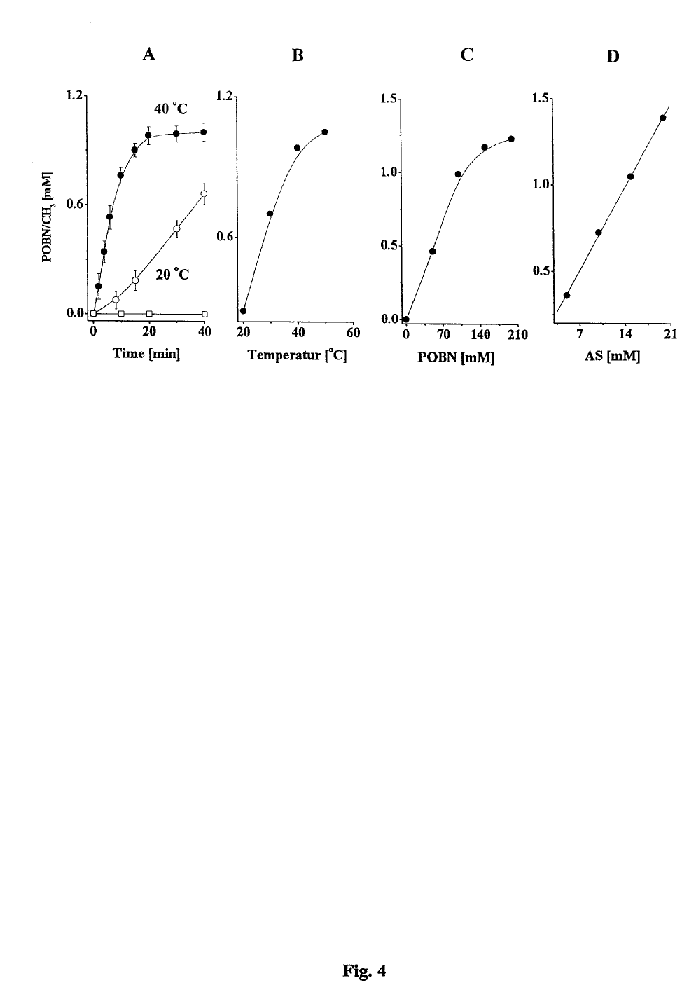

An addition of K₃[Fe(CN)₆] to a solution of AS, DMSO and PBN resulted in a transient increase of ESR signal of reaction Scheme III, 7, suggesting the occurrence of a Fe^{III}-dependent oxidation of reaction Scheme III, 10 back to 7. When the reaction solutions containing reaction Scheme III, 7 and 8 were analyzed by HPLC with electrochemical (EC) and/or UV detection, the predominant presence of the hydroxylamines of reaction Scheme III, 9 and 10 was observed (FIGS. 3B and C). The identity of compounds of reaction Scheme III, 7-10 was confirmed by co-injections of authentic standards, as well as via ESR and GC/MS (gas chromatograph coupled with mass spectrometer) analysis of the fractions defined by the corresponding HPLC peaks. Under the experimental conditions used, the generation of the composition of reaction Scheme III, 9 and 10 was well controlled and the yield of reaction Scheme El, 9 was approximately 10% from the initial AS concentration as set forth in FIG. 4.

FIG. 4 shows the optimization of the POBN-dependent spin trapping of .CH₃ in solutions of AS and DMSO. All reactions were carried out for either 40 minutes (Panels A and B) or 15 min (panels C and D) in 0.1 phosphate buffer (pH=5) containing DMSO (0.2 mM), POBN (panels A, B and D, 0.1 M) and AS (panels A-C, 15 mM). Panels A and B depict the temperature effects on the formation of POBN/CH₃ hydroxylamine (A-open circles, 20° C.; closed circles, 40° C.); in the absence of AS. Formation of POBN/CH₃ was not observed (open squares). The data presented in panel C suggests that a maximal spin-trapping efficiency could be obtained at approximately 0.2 M POBN; under these experimental conditions, the concentration of AS did not affect the linearity of POBN/CH₃ formation (panel D). From the data in FIG. 4, it is believed that at least 10% of AS hydrolyzes to hydroxyl radical.

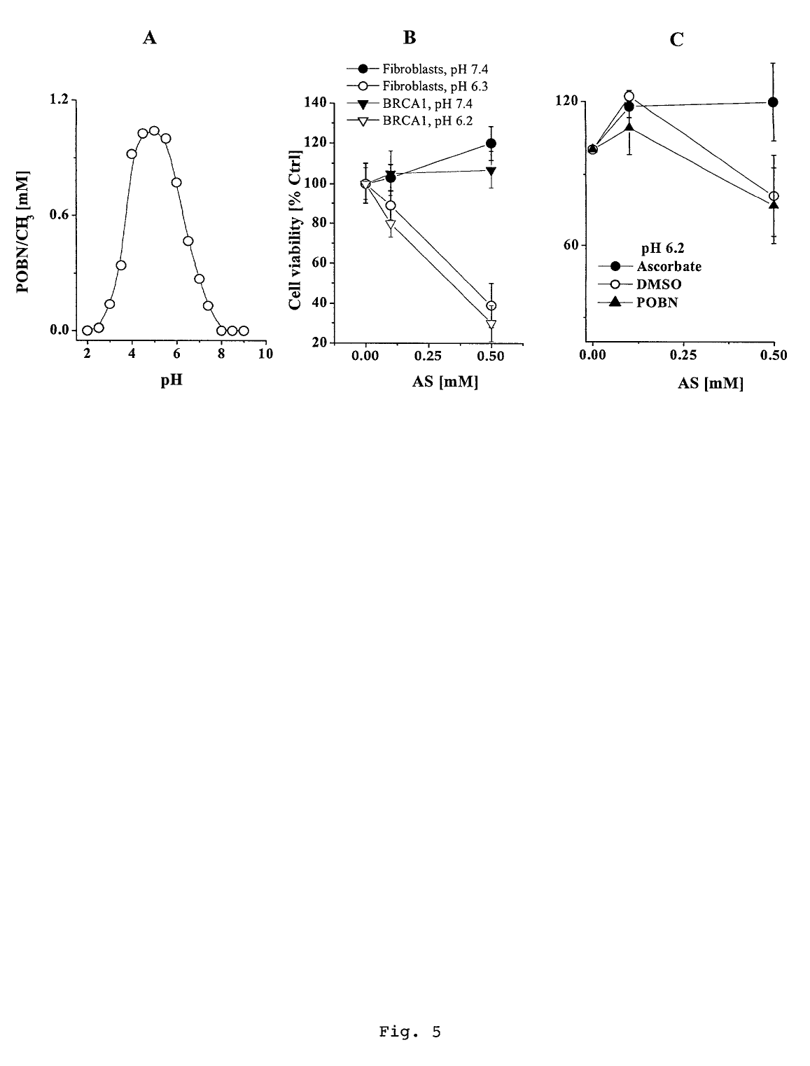

FIG. 5 shows the effects of the proton concentrations on the hydrolysis of AS to HO. and the AS-induced toxicity to normal human fibroblasts. Human fetal fibroblasts were cultured as described herein. Briefly, human fetal hearts of gestational age 16-24 weeks are aseptically obtained after elective termination of normal pregnancy by dilatation and evacuation. The aorta is then cannulated for continuous perfusion of the coronary arteries with calcium-free Tyrode's solution (117 mM NaCl, 5.7 mM KCl, 11 mM glucose, 4.4 mM NaHCO3, 1.5 mM KH2PO4, 1.7 mM MgCl2, HEPES 20 mM, pH 7.4) containing 1 U/ml of Na-heparin at 37° C., bubbled with 100% O2 as described for the Langendorff preparation. After 15 min of washing to clear the blood from the heart, fresh calcium-free Tyrode's solution containing 1.5 mg/ml collagenase A (type III) is recirculated for approximately 20 minutes. The heart dissociates spontaneously, allowing cells to slowly drip and fall on a Petri dish containing 0.25% trypsin, 1 mM EDTA in HBSS. Clumps of cells are dissociated and the resulting suspension poured over a cell strainer. Cells are centrifuged and the pellet resuspended in 20 ml of culture medium [DME supplemented with 10% fetal bovine serum, 50 U/ml PCN, 50 U/ml streptomycin, 100 mg/ml gentamicin, 1 mM non-essential amino acid (Gibco Laboratories), 0.1 mM essential medium vitamins (Gibco Laboratories), 2 mM glutamine, 0.1 mM Na pyruvate]. To obtain primary cardiac fibroblasts, the isolate will be plated in flasks (20 min at 37° C.). The fibroblasts will be passaged at 12×106 cells per 75 cm2 culture flask and grown in 5% CO2 at 37° C. After 3 passages in culture, the fibroblast stain using the fibroblast-specific antibody (monoclonal anti-human fibroblast surface protein, Clone 1B10).

FIG. 5A shows the HPLC-UV analysis of POBN/CH₃ hydroxylamine formed in solutions of AS (15 mM), DMSO (0.2 M) and POBN (0.1 M) in 0.1 M phosphate buffer (at varying pH values) upon an incubation of 30 minutes at 37° C. The concentration of the hydroxylamine was determined by co-injection of 4-methylpicoline as described in Analytical Chemistry, Stoyanovsky, et al., supra. FIG. 5B shows the effects of the proton concentration on the AS-induced toxicity to normal human fibroblasts and breast cancer cells. Normal human fibroblasts (800 cells per plate) were treated for 30 minutes at 37° C. with AS in 50 mM phosphate buffer (pH 6.3, open circles; pH 7.4, filled circles) containing 0.15 M NaCl and 0.2 mM CaC12. Thereafter, the fluid was removed, the cells were covered with minimal essential medium containing 10% fetal bovine serum (Sigma Chem. Co.,St. Louis, Mo.) and incubated at 37° C.; after 4 hours, the cell number was determined by the crystal violet method as known by those skilled in the art. Results are from four independent experiments. Each experimental point represents the mean of triplicate±SEM % (Mean value±standard error; n=3) of controls. FIG. 5C shows the effects of the proton concentration on the AS-induced toxicity to normal human fibroblasts. The cells were treated as indicated in FIG. 5B except that in the incubation medium was included (1) ascorbic acid (5 mM) plus SOD (superoxide dismuthase)(300 U per ml) plus catalase (500 U per ml) filled circles, all purchased from Sigma Chem. Co.; (2) POBN (10 mM) open circles; or 3. DMSO (0.2 mM) filled triangles.

In contrast to the AS-dependent generation of NO., the hydrolysis of AS to HO. was proton-dependent (FIG. 5A), suggesting the existence of a pH optimum for the formation and/or decomposition of H₂N₂0₂ (Scheme II). A maximal production of HO. was obtained within the pH interval of 4 to 6, which remarkably coincides with the pH of actively glycolyzing tumors. At pH 5.5 the AS-dependent production of HO. was 43 times higher than that at pH 7.4 (FIG. 5A), thus evidencing that AS is more toxic to cells with acidic extracellular pH. This was confirmed in experiments with normal human fibroblasts (closed circles, pH 7.4; open circles, pH 6.3) and breast cancer cells (closed triangles, pH 7.4; open triangles, pH 6.2; BRCA1, OMIM Number 113705, Coriell Institute For Medical Research, Camden, N.J.) (FIG. 5B). At pH 6.2, AS caused the death of approximately 70% of the treated cells, while at pH 7.4 cell toxicity was not observed. This cytotoxicity is dependent on the production of HO. as suggested by the protective effect of ascorbic acid (5 mM), POBN (10 mM) and DMSO (0.2 M) on human fibroblasts; ascorbate, POBN and DMSO are efficient scavengers of HO.. The comparable effect of AS on the studied cells suggests that the extracellular pH is the determining toxicological factor, rather than the cellular phenotype. A direct interaction of DMSO and POBN with AS and NO. was ruled out, as increased concentrations of DMSO (data not shown) and POBN lead to more efficient spin trapping of the HO.-generated .CH3 (FIG. 4C; reaction Scheme III, 3 to 6 to 8).

Scheme IV depicts the synthesis of Piloty's acid (PA), which is known to release NO⁻ upon hydrolysis in aqueous solutions (Scholz, J. N.; Engel, P. S.; Gildwell, C.; Whitmire, K. H.. 1989 Tetrahedron 45, 7695-7708; Zamora, R.; Grzesiok, A.; Wber, H.; Feelisch, M. 1995 Biochem. J. 312, 333-9):

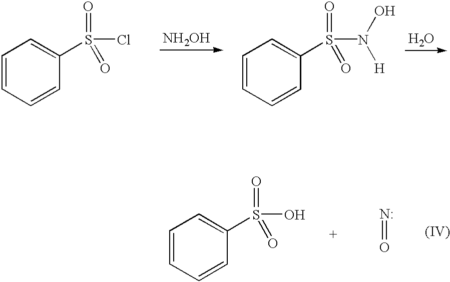

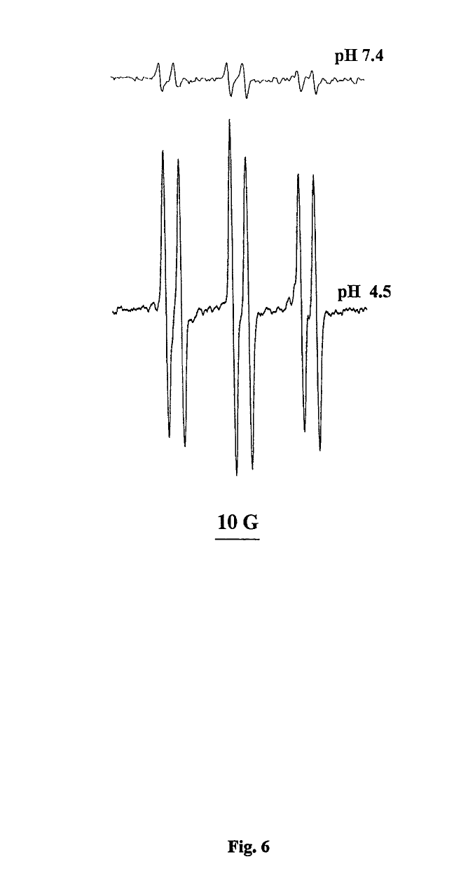

When the hydrolysis of PA (0.5 mM) was carried out in 0.1 phosphate buffer (varying pH) in the presence of PBN (0.02 M) and DMSO (0.2 M), the typical ESR spectrum of PBN/CH₃ could be observed (FIG. 6). The latter suggests the occurrence of the reaction sequence presented in Scheme I. Similarly to AS, the hydrolysis of PA in acidic solutions was paralleled by more intensive generation of hydroxyl radical.

Examples of organic compounds with activated —N—O— functions that hydrolyze (as PA and AS) with release of NO⁻ (respectively HO.) are presented below:

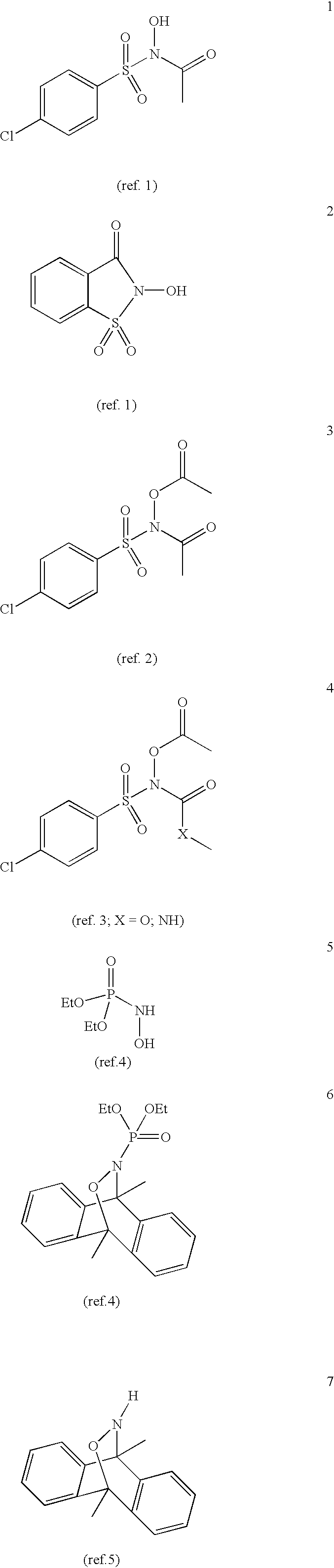

1. N-Acetyl-4-chloro-N-hydroxy-benzenesulfonamide; 2. 2-Hydroxy-1,1-dioxo-1,2-dihydro-116-benzo[d]isothiazol-3-one; 3. N-Acetoxy-N-acetyl-4-chloro-benzenesulfonamide; 4. derivatives of N-Acetoxy-4-chloro-benzenesulfonamide (X=0; NH); 5. N-hydroxy-diethoxysulfonamide; 6. and 7. 2-Oxa-3-aza-bicyclo[2.2.2]octane modified in 1st, 4th, 5th, 6th, 7th, and/or 8^{th} position(s), as well as via acylation of its —NH— function.

References (ref.)

1. Lee, M. J. C.; Elberling, J. A.; Nagasawa, H. T. 1992 J. Med. Chem. 35, 3641-47.

2. Ware, R. W.; King, S. B. 2000 J. Org. Chem. 65, 8725-8729.

3. Ensley, H. E.; Mahadevan, S. 1989 Tetrahedron Lett. 30, 3255-58.

4. Nagasawa, H. T.; Kawle, P. S.; Elberling, J. A.; Demaster, E. G.; Fukoto, J. M. 1995 J. Med. Chem. 38, 1865-71.

5. Lee, M. J. C.; Nagasawa, H. T.; Elberling, J. A.; DeMaster, G. 1992 J. Med. Chem. 35, 3648-52.

It will be appreciated by those skilled in the art that the present invention provides a method of treating patients having a cancerous disease employing compositions and pharmaceutically acceptable salts that are capable of generating a hydroxyl radical wherein the extracellular pH of the cancer cell(s) are less than 7.0. Hydroxyl radical is a highly toxic species that interacts indiscriminately, in a diffusion-controlled manner with low molecular weight compounds, lipids and proteins. AS exhibits a distinct toxicity to wide array of cancer cells that are surrounded by an acidic microenvironment.

It will be understood by those skilled in the art that the present method shall inhibit cancerous cell growth in a patient.

Whereas particular embodiments of this invention have been described herein for purposes of illustration, it will be evident to those skilled in the art that numerous variations of the details of the present invention may be made without departing from the invention as defined in the appended claims.

We claim:

1. A method for treating a patient having a cancerous disease comprising administering to the patient an effective amount of a composition capable of generating a hydroxyl radical at an extracellular pH at or less that about 7.0.

2. The method of claim 1 including administering said composition to the patient wherein the extracellular pH of the cancer cell is from about 3.5 to 7.0.

3. The method of claim 1 including administering to the patient said composition wherein said composition is sodium trioxodinitrate (Angeli's salt).

4. The method of claim 1 including administering to the patient said composition wherein said composition is an N-substituted hydroxylamine.

5. The method of claim 1 including administering to the patient said composition wherein said composition is Pyloti's acid.

6. The method of claim 1 including administering to the patient said composition wherein said composition has the formula X—N(Y)—OH, wherein X is an electron withdrawing group, and wherein Y is one or more of the groups selected from —H, —O—, CH₃CO—, and —CO—O′—CO—NH—.

7. The method of claim 6, wherein X is selected from the group consisting of —H, —NO₂, (EtO)₂P(O)—, —SO₂—, and C₆H₅SO₂.

8. The method of claim1 wherein said composition prior to generating said hydroxyl radical is capable of generating a nitroxyl anion

9. The method of claim 1 including incorporating said composition in a suitable pharmaceutical carrier and administering a therapeutically effective amount of said composition incorporated into said pharmaceutical carrier to said patient.

10. The method of claim 1 including employing said method in prophylactically treating a patient to provide protection against a cancerous illness.

11. The method of claim 9 including administering said composition incorporated in said pharmaceutical carrier to the patient by the parenteral route.

12. The method of claim 9 including administering said composition incorporated into said pharmaceutical carrier to the patient by the oral route.

13. The method of claim 9 including administering said composition incorporated into said pharmaceutical carrier to the patient topically.

14. The method of claim 9 including employing said composition incorporated into said pharmaceutical carrier to the patient by injection into the location of the cancerous disease.

15. The method of claim 9 including employing said pharmaceutical carrier comprising physiologic saline.

16. The method of claim 9 including employing said pharmaceutical carrier comprising 5% dextrose for injection.

17. The method of claim 9 including employing said pharmaceutical carrier comprising 5% NaHCO₃ for injection.

18. The method of claim 9 including employing said pharmaceutical carrier comprising physiologic saline, 5% dextrose for injection, 5% NaHCO₃ for injection, and combinations thereof.

19. The method for inhibiting the growth of a cancerous tumor in a patient comprising administering to the patient a composition capable of generating a hydroxyl radical in a pH-dependent manner in an amount effective to inhibit the growth of the cancerous tumor.

20. The method of claim 19 including administering to the patient said composition wherein said composition is sodium trioxodinitrate.

21. The method of claim 19 including administering to the patient said composition wherein said composition is Pyloti's acid.

22. The method of claim 19 including administering to the patient said composition wherein said composition is an organic compound with activated —N—O— function(s) selected from the group consisting of N-substituted hydroxylamines, nitronates, esters of hydroxamic acids, P-nitrosophosphates, 2-Oxa-3-aza-bicyclo[2.2.2]octane derivatives, all of which hydrolyze with release of NO⁻.

23. The method of claim 19 including administering to the patient said composition wherein said composition has the formula X—N(Y)—OH , wherein X is selected from the group consisting of —H, —NO₂; (EtO)₂P(O)—; —SO₂—, and C₆H₅SO₂, and Y is selected from the group consisting of —H, —O—, CH₃CO—, and —CO—O—′—CO—NH—.

24. The method of claim 19 including administering to the patient said composition wherein said composition has the formula X—NH—OH wherein X is an electron-withdrawing group.

25. The method of claim 24 including wherein X is selected from the group consisting of N0₂ and C₆H₅S0₂.

26. The method of claim 1 including wherein X is selected from at least one of the group consisting of N0₂ and C₆H₅S0₂, and Y is H.

27. The method of claim 1 including wherein said composition is nitric oxide with co-administration of nitric oxide-reducing agents that generate NO⁻.

28. The method of claim 19 including wherein said composition is nitric oxide with co-administration of nitric oxide-reducing agents that generate NO⁻.

29. The method of claim 1 wherein said composition has the formula R₂C═N(O)—OH, wherein R is selected from the group consisting of alkyl and aryl residues.

30. The method of claim 19 wherein said composition has the formula R₂C═N(O)—OH, wherein R is selected from the group consisting of alkyl and aryl residues.

31. The method of claim 1 wherein said composition is hyponitrous acid.

32. The method of claim 19 wherein said composition is hyponitrous acid.
